(19) United States
(12) Patent Application Publication
(10) Pub. No.: US 2024/0398402 A1
(43) Pub. Date: Dec. 5, 2024

(54) SYSTEMS AND METHODS FOR PLACEMENT OF MULTIPLE JOINED SUTURE ANCHORS

(71) Applicant: ACTIVAS Orthopedics, Inc., Coronado, CA (US)

(72) Inventors: Colin Murphy, Carlsbad, CA (US); Brian Bowman, Carlsbad, CA (US); Benjamin Arnold, San Diego, CA (US); Thomas A. Afzal, Coronado, CA (US)

(21) Appl. No.: 18/678,223

(22) Filed: May 30, 2024

Related U.S. Application Data
(60) Provisional application No. 63/469,649, filed on May 30, 2023.

(51) Int. Cl. A61B 17/04; A61F 2/08

(52) U.S. Cl. CPC A61B 17/0401; A61F 2/0811; A61B 2017/0409; A61B 2017/0464; A61F 2002/0882

(57) ABSTRACT

Systems, devices, and methods for deploying a graft to a treatment site of a patient. Exemplary systems operate to fix a graft to bone or soft tissue by placing multiple anchors through the graft in different locations, with individual anchors interconnected by sutures fixing the graft to the patient tissue. Exemplary anchor assemblies include an initial anchor with a suture permanently affixed, intermediate anchors through which the running suture can pass freely after placement, and a final anchor which locks the running suture under tension once placed.

### CROSS-REFERENCES TO RELATED APPLICATIONS

This application claims the benefit of U.S. Provisional Patent Application No. 63/469,649 filed May 30, 2023, the disclosure of which is incorporated herein by reference.

### BACKGROUND

Embodiments of the present invention relate generally to implant deployment systems and methods, and in particular instances, to implant deployment systems and methods for placing multiple joined suture anchors.

Many available suture methods require complicated routing and fixation techniques, which can increase the probability of error in a surgical procedure. In some instances, currently available devices must cross the skin barrier multiple times during a procedure, which can increase the risk of infection.

Likewise, many currently available techniques for providing soft tissue to bone fixation require multiple instruments and complex suture management to ensure that all parts are properly fixated. Often, additional instruments and procedures are further required in order to apply the suture to the graft for the procedure. Moreover, typically known devices require a separate instrument for bore creation prior to placement of an anchor in bone.

Hence, although current implant deployment modalities enable surgeons to provide beneficial treatments to patients in need thereof, still further improvements in implant deployment technology are desired. Embodiments of the present invention provide solutions to at least some of these outstanding needs.

### BRIEF SUMMARY

Exemplary graft deployment systems or devices as disclosed herein can be provided as a single use device for the deployment of an implant for surgical repair or reconstruction. In some cases, systems and methods can be used to fix soft tissue and/or graft material to a patient bone tissue. Exemplary embodiments can simplify procedures by packaging full functionality into a single-use instrument, reducing costs associated with reprocessing. In some embodiments, devices can remove the need to route one or more sutures, thus reducing the complexity of procedures and reducing the chance for error. Exemplary device and method embodiments can use a cartridge that allows for the ability to rapidly multiple anchors consecutively, resulting in shorter procedures. Use of device and method embodiments disclosed herein can greatly reduce the number of times an instrument must cross the skin barrier during the procedure, thus reducing the risk of infection for a patient.

In one aspect, embodiments of the present invention encompass systems and methods for treating a bone or soft tissue of a patient. Exemplary systems can include a graft, an anchor assembly, and a deployment device. An anchor assembly can include a first anchor, a second anchor, and a suture in operative association with the first anchor and the second anchor. A deployment device can be configured to deliver the anchor assembly to the bone or soft tissue of the patient. In some cases, the deployment device is configured to store the anchor assembly prior to delivering the anchor assembly to the bone or soft tissue of the patient. In some cases, the deployment device includes a sheath (e.g. outer sheath) that is configured to remain in an incision or port of the patient until each anchor of the anchor assembly is placed in the bone or soft tissue of the patient. In some cases, the anchor assembly includes a third anchor and a fourth anchor, and the suture is in operative association with the third anchor and the fourth anchor. In some cases, at least one anchor of the anchor assembly does not pass through the graft. In some cases, at least one anchor of the anchor assembly is an expandable anchor. In some cases, at least one anchor of the anchor assembly is made of or includes polyether ether ketone. In some cases, at least one anchor of the anchor assembly is an all-suture anchor. In some cases, the deployment device includes a strike surface configured for malleting. In some cases, the deployment device includes a spring loaded cartridge. In some cases, the deployment device includes an awl. In some cases, each anchor of the anchor assembly is configured to be inserted on an awl. In some cases, each anchor of the anchor assembly is self-awling. In some cases, each anchor of the anchor assembly is configured to be stored in a revolving barrel or cartridge. In some cases, the deployment device includes a first insertion shaft and a second insertion shaft that is parallel with the first insertion shaft. In some cases, the first anchor is configured to releasably engage with the first insertion shaft and the second anchor is configured to releasably engage with the second insertion shaft.

In another aspect, embodiments of the present invention encompass anchor assemblies and methods for treating a bone or soft tissue of a patient. Exemplary anchor assemblies can include a first or initial anchor, a suture affixed with the first or initial anchor, one or more intermediate anchors configured to receive the suture therethrough, and a final anchor configured to lock the suture under tension once placed. In some cases, the first or initial anchor includes a suture path, one or more outer fixation mechanisms, a cannula, and a driver connection mechanism. In some cases, at least one of the intermediate anchors includes a suture path, one or more outer fixation mechanisms, a cannula, and a driver connection mechanism. In some cases, the final anchor includes a suture path, one or more outer fixation mechanisms, a cannula, and a driver connection mechanism. In some cases, at least one of the anchors is an expandable anchor. In some cases, the first or initial anchor and the final anchor are expandable anchors.

### BRIEF DESCRIPTION OF THE DRAWINGS

Inventive features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates aspects of an anchor deployment device, according to embodiments of the present invention;

FIG. 2 illustrates aspects of an anchor deployment device, according to embodiments of the present invention;

FIG. 3 illustrates aspects of an anchor deployment device, according to embodiments of the present invention;

FIG. 4 illustrates aspects of an anchor deployment device, according to embodiments of the present invention;

FIG. 5 illustrates aspects of an anchor deployment device, according to embodiments of the present invention;

FIG. 6 illustrates aspects of a cartridge of an anchor deployment device, according to embodiments of the present invention;

FIG. 7 illustrates aspects of a cartridge of an anchor deployment device, according to embodiments of the present invention;

FIG. 8 illustrates aspects of a cartridge of an anchor deployment device, according to embodiments of the present invention;

FIG. 9 illustrates aspects of a cartridge of an anchor deployment device, according to embodiments of the present invention;

FIG. 10 illustrates aspects of a cartridge of an anchor deployment device, according to embodiments of the present invention;

FIG. 11 illustrates aspects of a cartridge of an anchor deployment device, according to embodiments of the present invention;

FIG. 12 illustrates aspects of a cartridge of an anchor deployment device, according to embodiments of the present invention;

FIG. 13 illustrates aspects of a cartridge of an anchor deployment device, according to embodiments of the present invention;

FIGS. 14A to 14F illustrate aspects of an anchor and a suture of a deployment system, according to embodiments of the present invention;

FIGS. 15A to 15C illustrate aspects of an anchor of a deployment system, according to embodiments of the present invention;

FIGS. 16A to 16D illustrate aspects of an anchor and a suture of a deployment system, according to embodiments of the present invention;

FIGS. 17A and 17B depict aspects of a cartridge of an anchor deployment device, according to embodiments of the present invention;

FIGS. 18A to 18D illustrate aspects of an anchor deployment system, according to embodiments of the present invention;

FIGS. 19A to 19I illustrate aspects of an anchor deployment system, according to embodiments of the present invention;

FIGS. 20 and 21 depict aspects of a cartridge of an anchor deployment device, according to embodiments of the present invention;

FIGS. 22 and 23 depict aspects of an anchor deployment device, according to embodiments of the present invention;

FIGS. 24 to 32 depict aspects of an anchor deployment device and procedure, according to embodiments of the present invention;

FIG. 33 illustrates aspects of an anchor deployment system, according to embodiments of the present invention;

FIG. 34 illustrates aspects of an anchor deployment system, according to embodiments of the present invention;

FIG. 35 illustrates aspects of an anchor deployment system, according to embodiments of the present invention;

FIG. 36 illustrates aspects of an anchor deployment system, according to embodiments of the present invention;

FIG. 37 illustrates aspects of an anchor deployment system, according to embodiments of the present invention;

FIG. 38 illustrates aspects of an anchor deployment system, according to embodiments of the present invention;

FIG. 39 illustrates aspects of an implant, according to embodiments of the present invention;

FIGS. 40 to 48 illustrate aspects of an implant procedure, according to embodiments of the present invention;

FIG. 49 illustrates aspects of an anchor deployment system and procedure, according to embodiments of the present invention;

FIG. 50 illustrates aspects of an anchor deployment system and procedure, according to embodiments of the present invention;

FIG. 51 illustrates aspects of an anchor deployment system and procedure, according to embodiments of the present invention;

FIG. 52 illustrates aspects of an anchor deployment system and procedure, according to embodiments of the present invention;

FIG. 53 illustrates aspects of an anchor deployment system and procedure, according to embodiments of the present invention;

FIG. 54 illustrates aspects of an anchor deployment system and procedure, according to embodiments of the present invention;

FIG. 55 illustrates aspects of an anchor deployment system and procedure, according to embodiments of the present invention; and

FIG. 56 illustrates aspects of an anchor deployment system and procedure, according to embodiments of the present invention.

### DETAILED DESCRIPTION

Specific embodiments of the disclosed device, system and method of use will now be described with reference to the drawings. Nothing in this detailed description is intended to imply that any particular component, feature, or step is essential to embodiments of the invention.

Exemplary fixation systems and methods disclosed herein can include or involve a series of single use devices for the fixation of graft or other soft tissue to bone or other soft tissue. In some embodiments, a system includes a fixation device, multiple anchors, and a length of suture. In some embodiments, a fixation device includes an anchor connection, a shaft, a cartridge, an awl, an outer sheath, a resetting arm, and a handle. Exemplary anchors can include a driver connection, a cannula or an awl, a suture path, and outer fixation features. An exemplary cartridge of a fixation device can operate to hold multiple anchors, and the anchors can be internally interconnected. In some instances, anchors can be driven into bone or soft tissue with the actuation of a shaft, and the fixation device can then load the next anchor onto the shaft such that multiple interconnected anchors can be placed in series from the single fixation device.

In some embodiments, a system can include a fixation device, one or more anchors, and a length of suture. An exemplary fixation device can include an anchor connection, a shaft, a cartridge, an awl, an outer sheath, a resetting arm, and a handle. In some instances, the anchor connection is located on the distal end of the shaft, with the awl extending from it. The handle can contain the cartridge and resetting arm, and can be attached to the outer sheath on its distal side. In some embodiments, the outer sheath features a suture path so that a suture has space to move next to the anchor. In some embodiments, the shaft is constrained such that it can move axially through the handle and outer sheath. In some cases, the shaft includes a mechanism which interacts with the resetting arm in order to move the arm as the shaft is moved back. In some embodiments, a cartridge or cartridge assembly can include a cartridge body, one or more cannulas for anchor storage, one or more suture routing pins, one or more suture paths, and one or more rotation stops. In some cases, a cartridge can be spring-loaded such that it cannot rotate while all rotation stops are in place. In some cases, a cartridge can be configured to rotate a prescribed distance to the next rotation stop once a prior rotation stop is moved.

In some embodiments, an anchor can include a driver connection, a cannula or an awl, a suture path, and one or more outer fixation features. In some cases, an outer fixation feature can be provided as one or more barbs. In some embodiment, a first anchor to be placed includes a suture attachment feature which attaches it to the length of suture, either before or during insertion of the anchor into its destination, and the last anchor to be placed includes a suture locking feature which attaches it to the length of suture during or after the insertion of the anchor into its destination.

In some embodiments, a length of suture has a fixed end, fixed to the first anchor to be placed, and a free end which can be tensioned.

In some embodiments, an anchor can be initially loaded on the distal end of the shaft, with the anchor connection connected to the driver connection and the awl extending fully through the anchor. A single length of suture can be run through all the anchors' suture paths in series such that they may move freely over the suture in the initial configuration. The length of suture can be placed through the cartridges suture paths between the anchors. In operation, the shaft can be driven forward to drive the anchor into its target location.

The first placed anchor can include a feature or mechanism which prevents the suture from being pulled free once it is inserted. After the first anchor is placed, the fixation device can be moved, with the length of suture running from the now placed anchor into the fixation device and through the cartridge and the other anchors. The shaft can then be drawn back, causing the resetting arm to move the current rotation stop and free the cartridge to revolve and align the next anchor with the shaft. The shaft can then be run forward, engaging with the next anchor and placing the anchor once driven fully forward. The length of suture can be looped through the anchors and cartridge such that it comes free of the cartridge as the anchors are placed. This process may be assisted by the release of the suture by the pulling of a suture routing pin. The length of suture can thus be routed between each of the sutures placed such that it is loose, able to move axially freely through the intermediate anchors, while maintaining its path through the suture paths of the anchors.

Any desired number of the intermediate sutures can be placed through the soft tissue to be fixated such that the suture connecting the anchors to each other will fixate the soft tissue once the suture is tensioned. The anchor placement process can be repeated until the final anchor is ready to be placed. At this point the free end of the suture length can be tensioned to tighten the suture between the anchors. The final anchor can then be placed and the suture locking feature can be engaged. The resulting configuration can have the length of suture routed in a stitch pattern between the inserted anchors and provides fixation to the soft tissue.

In some embodiments, a shaft or knob can include a strike or distal surface for malleting. In some embodiments, a device can include a knob for manipulating the shaft by hand. In some embodiments, a device can include a button for pulling the suture routing pins. In some embodiments, a device can include a suture knob around which excess suture can be looped. In some cases, a suture pulling loop can be attached to the free end of the suture to assist in tensioning the suture. In some cases, a system can include a tool for cutting excess suture after the last anchor placement. In some cases, an anchor can include a polymer material such as polyether ether ketone (PEEK). In some cases, an anchor can include a material such as all-suture, resorbable metal, resorbable collogen, nitinol, or the like. In some cases, an anchor can have a size within a range from about 3 mm to about 4.5 mm in diameter.

According to exemplary embodiments, an awl in a fixation device allows for the rapid placement of anchors without the need for additional instrumentation. In exemplary embodiments, a cartridge and quick/automatic reloading of a fixation device allows for the rapid placement of multiple anchors, thus accelerating the speed of procedures. In exemplary embodiments, placement of the anchors directly through soft tissue removes the need for complex suture management, thus simplifying procedures.

According to exemplary embodiments, an all-in-one disposable tool can simplify procedure process and remove the need for instrument reprocessing. In some embodiments, a suture run between multiple anchors can fixate the soft tissue in patterns as desired and without the need for soft tissue preparation or suture management.

According to exemplary embodiments, by combining an awl and anchor placer and preloading the suture through the anchor, it is possible to reduce the number of instruments required for the procedure. In some embodiments, an all-in-one disposable design can reduce the number of packages required for a case.

In some embodiments, the shaft can be actuated by hand. In some embodiments, the shaft can be actuated by stored energy. In some embodiments, the shaft can be actuated by pneumatics. In some embodiments, the length of suture can be or include suture tape. In some embodiments, the length of suture can be or include round suture. In some embodiments, one or more anchors can be made of or include PEEK. In some embodiments, one or more anchors can be all-suture anchors. In some embodiments, one or more anchors can be made of or include expanding nitinol. In some embodiments, a cartridge can be a rotary cartridge. In some embodiments, a cartridge can be a linear cartridge.

In some embodiments, a cartridge can be actuated by a shaft motion. In some embodiments, a cartridge can be actuated by a spring loaded mechanism. In some embodiments, a cartridge can include one or more suture routing pins. In some embodiments, a cartridge can have no suture routing pins. In some embodiments, a cartridge can be replaceable. In some embodiments, movement of the suture routing pins can be manually performed. In some embodiments, movement of the suture routing pins can be actuated by the shaft. In some embodiments, tensioning of the length of suture may be performed by the device. In some embodiments, the length of suture can be tensioned between each individual anchor. In some embodiments, the length of suture can be tensioned across the system or implant during the last anchor placement.

Implant deployment systems disclosed herein can be used to deploy implants to any of a variety of patient treatment sites. In some cases, an implant deployment system can be used to deploy a graft to a rotator cuff of a patient.

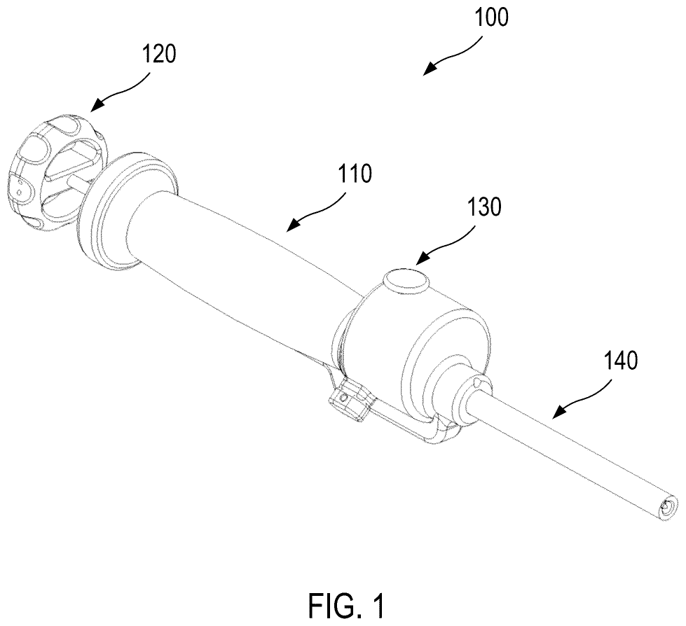

Turning now to the drawings, FIG. 1 depicts aspects of a fixation system or device 100, according to embodiments of the present invention. As shown here, fixation device 100 includes a proximal knob 120, a handle 110, a suture knob 130, and an outer sheath 140.

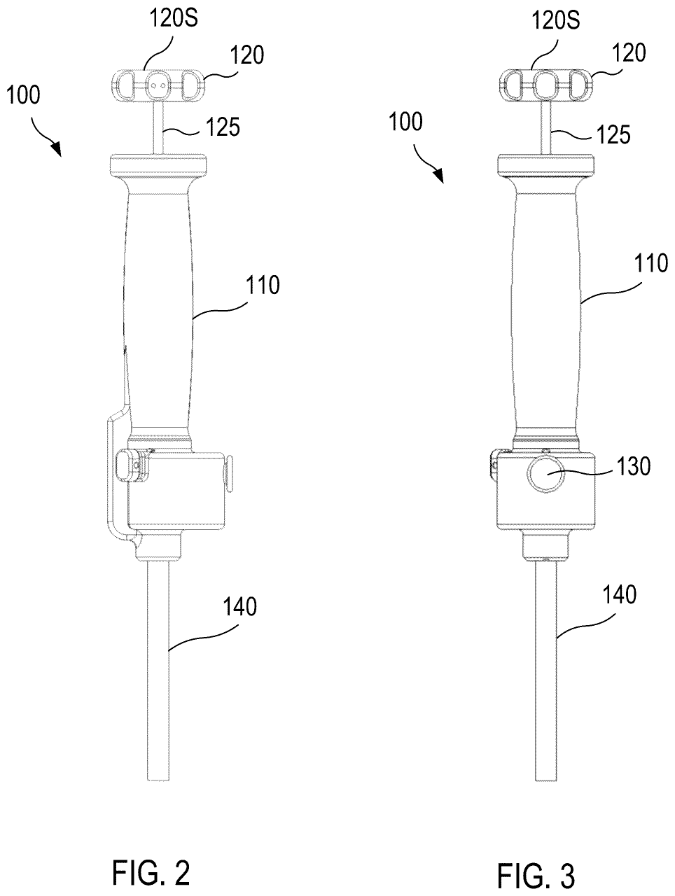

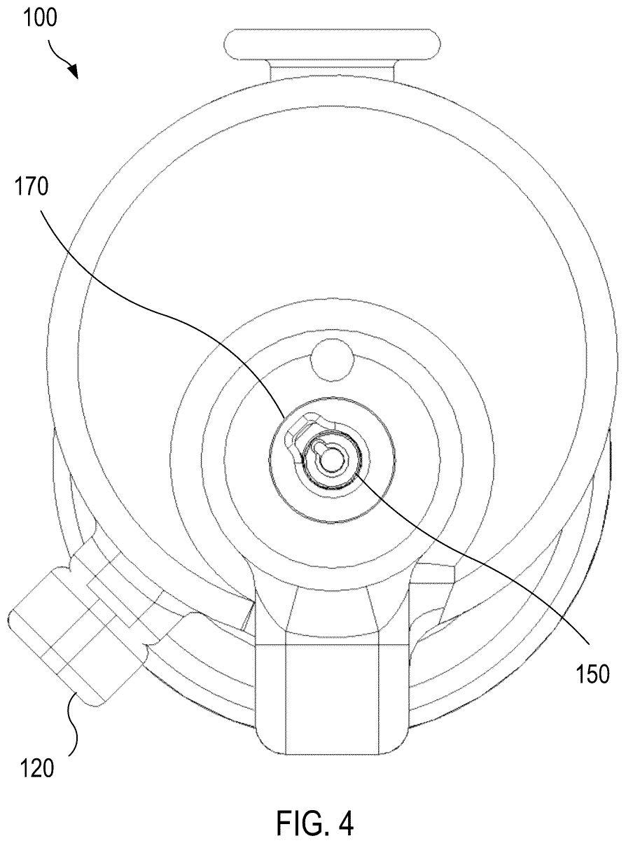

FIGS. 2 and 3 depict aspects of a fixation device 100 in an initial position. As shown here, fixation device 100 includes a proximal knob 120, a handle 110, a suture knob 130, and an outer sheath 140. Device 100 also includes a shaft 125 coupled with the knob 120. There is a striking surface 120S on a proximal side of the knob 120.

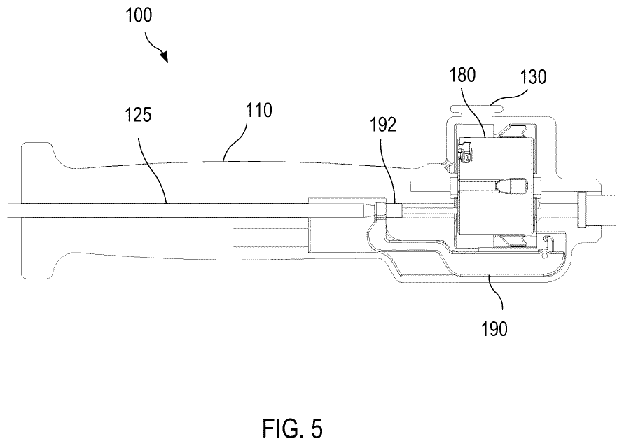

As shown in FIG. 4, fixation device 100 includes an anchor 150 and a button 160, and provides an outer sheath suture path 170.

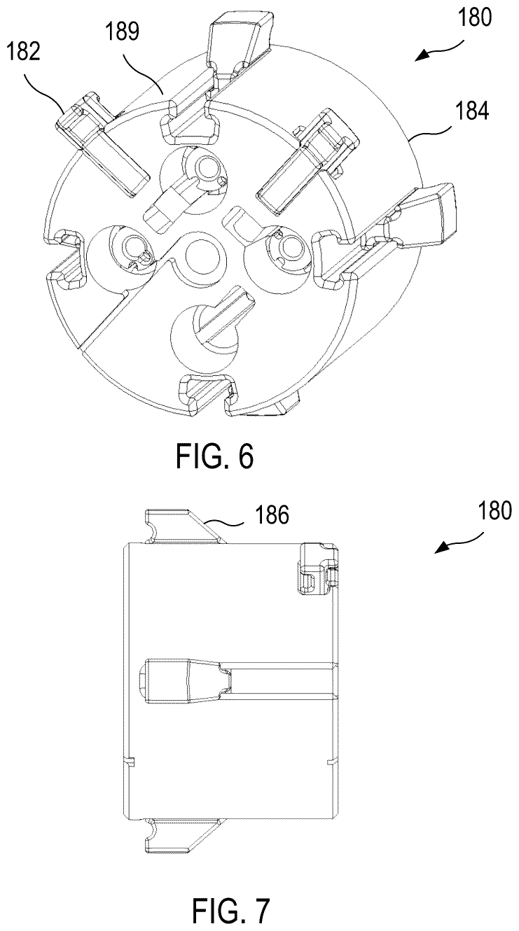

FIG. 5 provides a cross-section view of aspects of a fixation device 100 in an initial position. As shown here, device 100 includes a shaft 125, a handle 110, a suture knob 130, a cartridge 180, a resetting arm 190, and a resetting component 192. In operation, the resetting component 192 can interact with or engage the resetting arm 190 in order to move the resetting arm 190 in the proximal direction when the shaft 125 is moved in the proximal direction.

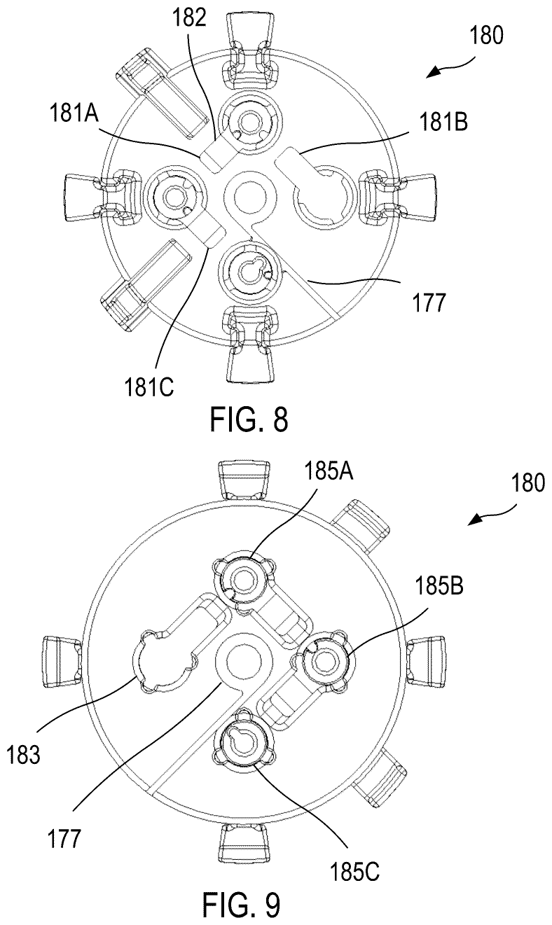

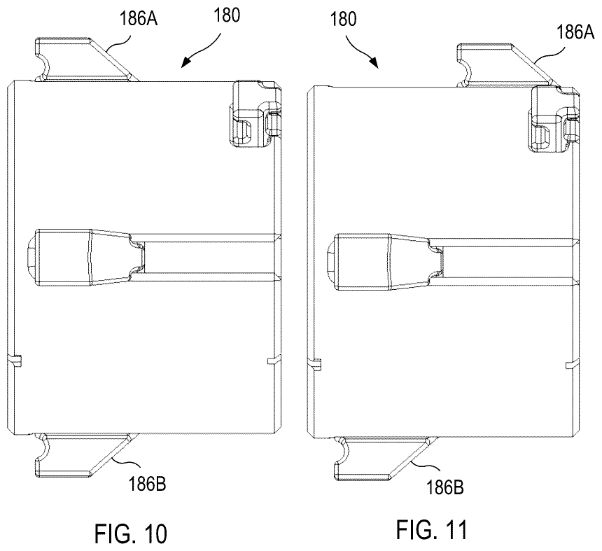

FIGS. 6 and 7 depict aspects of a cartridge 180, according to embodiments of the present invention. As shown here, cartridge 180 can include a suture routing pin 182, a cartridge body 184, and one or more rotation stops 186. In some embodiments, a cartridge can be configured to remain in an incision of the patient until one or more anchors of an anchor assembly are placed in the bone or soft tissue of the patient. In some cases, a cartridge can be referred to as a rotating barrel, and each anchor of an anchor assembly is configured to be stored in or engaged with the revolving barrel. In some embodiments, the suture routing pin 182 as depicted FIG. 6 can extend from the geometric outer body 189 of the cartridge 180.

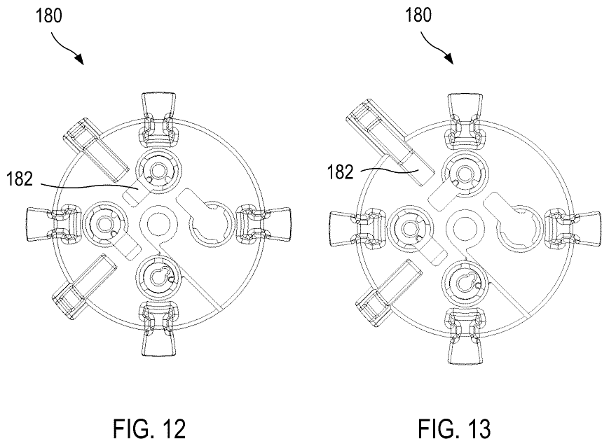

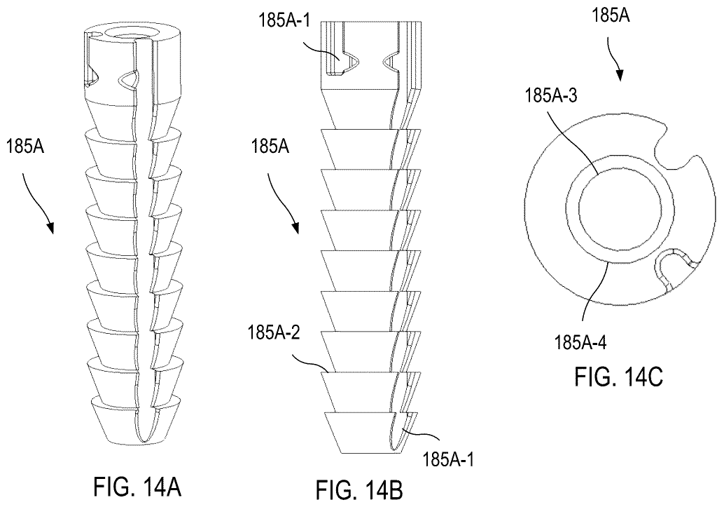

FIGS. 8 and 9 depict aspects of a cartridge 180, according to embodiments of the present invention. As shown here, cartridge 180 includes a suture routing pin 182 and provides a suture path 181A, 181B, 181C. Further, cartridge 180 includes a cannula 183, a first intermediate anchor 185A, a second intermediate anchor 185B, and a final anchor 185C. In some embodiments, the suture routing pin 182 as depicted in FIG. 8 can be at a cylindrical deeper body of the cartridge 180.

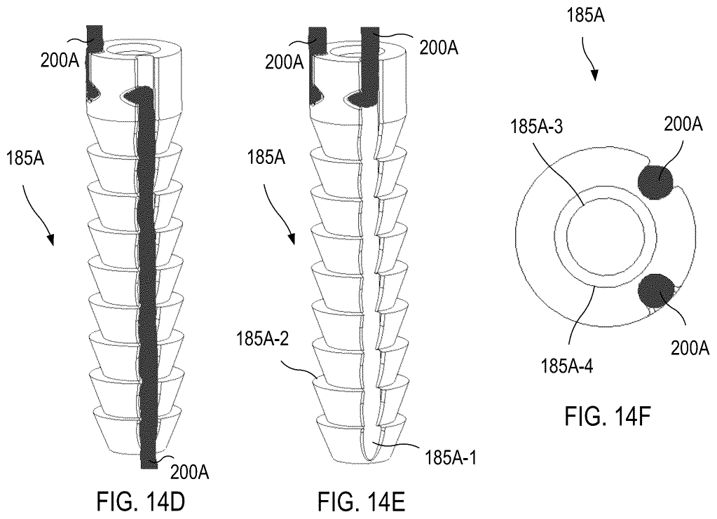

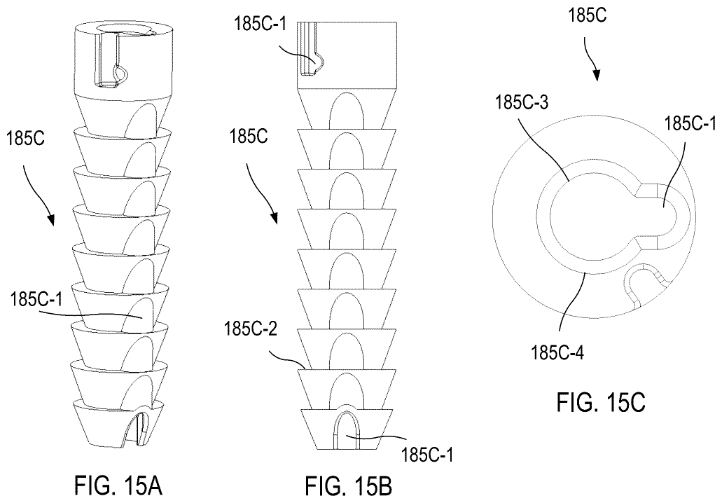

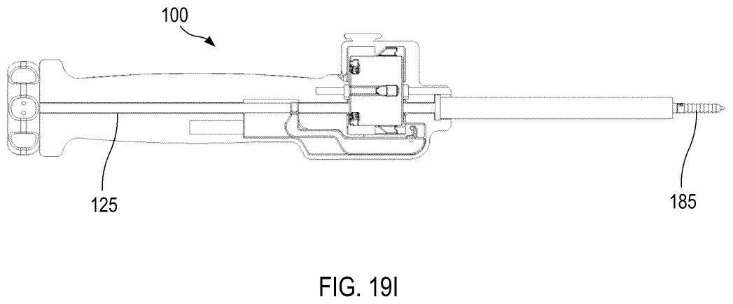

FIGS. 10 and 11 depict aspects of a cartridge 180, showing movement of rotation stops 186A and 186B. Movement of the rotation stops can be further understood with reference to the description of FIGS. 19A to 19I provided herein.

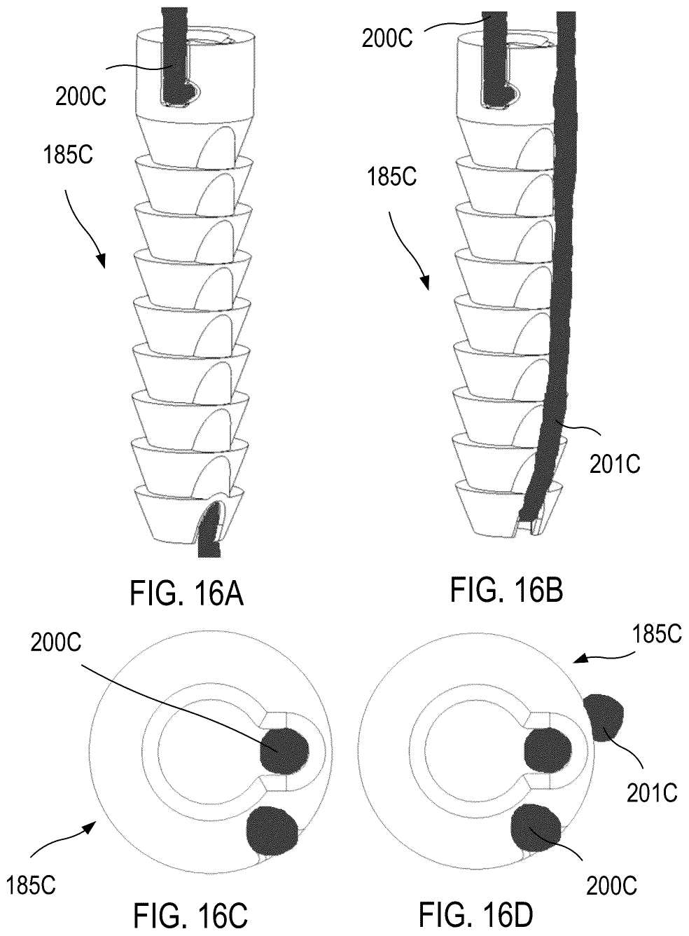

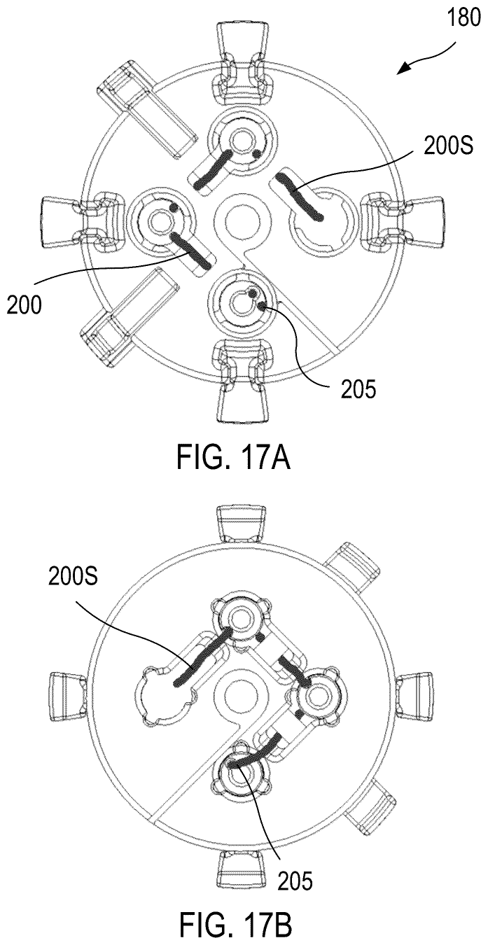

FIGS. 12 and 13 depict aspects of a cartridge 180, showing movement of suture routing pins. According to some embodiments, a suture routing pin can be moved radially out by the mechanism such that the suture routing pin no longer obstructs the corresponding suture path. FIG. 12 depicts the initial (e.g. advanced) suture routing pin 182 position and FIG. 13 depicts the system with the suture routing pin 182 in a retracted position.

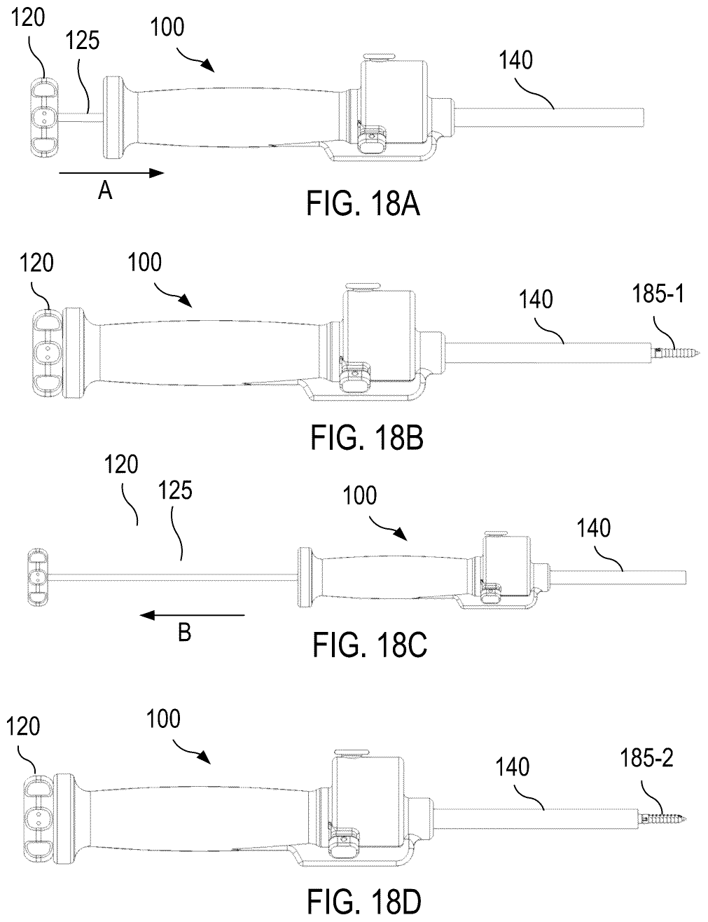

FIGS. 14A to 14C depict aspects of an intermediate anchor 185A, according to embodiments of the present invention. As shown here, intermediate anchor 185A includes a suture path 185A-1, one or more outer fixation mechanisms 185A-2, a cannula 185A-3, and a driver connection mechanism 185A-4.

FIGS. 14D to 14F depict aspects of an intermediate anchor 185A and a suture 200A, according to embodiments of the present invention. FIG. 14D depicts the suture 200A as it is routed in a pre-insertion configuration, and FIG. 14E depicts the suture 200A as it is routed in a post-insertion configuration. As shown here, intermediate anchor 185A includes a suture path 185A-1, one or more outer fixation mechanisms 185A-2, a cannula 185A-3, and a driver connection mechanism 185A-4.

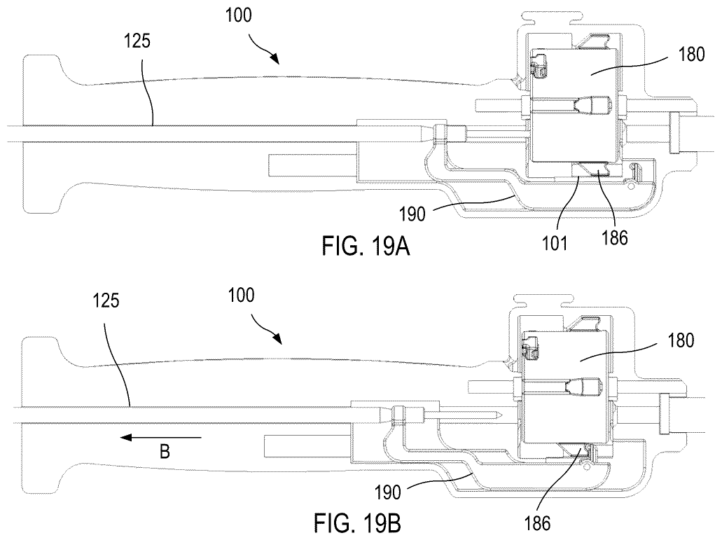

FIGS. 15A to 15C depict aspects of a final anchor 185C, according to embodiments of the present invention. As shown here, final anchor 185C includes a suture path 185C-1, one or more outer fixation mechanisms 185C-2, a cannula 185C-3, and a driver connection mechanism 185C-4.

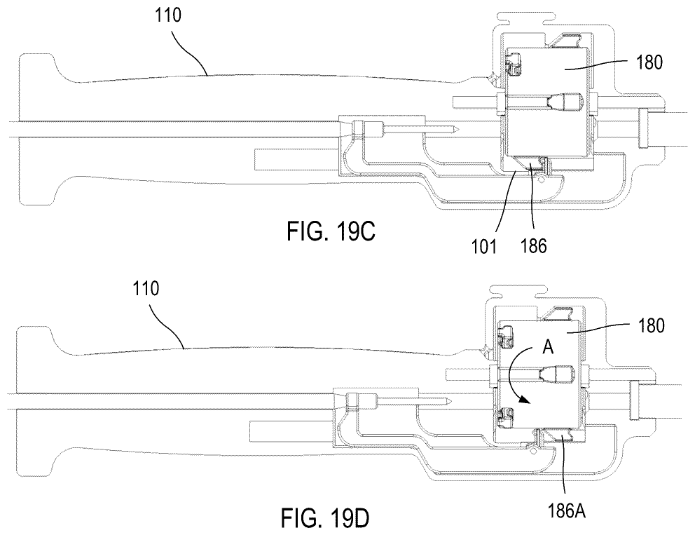

FIGS. 16A to 16D depict aspects of a first or final anchor 185C and a suture 200C, according to embodiments of the present invention. FIGS. 16A and 16C depict the suture 200C as it is routed in a pre-insertion configuration, and FIGS. 16B and 16D depict the suture 200C as it is routed in a post-insertion configuration. As shown here, in the post-insertion configuration, when a first or final anchor is placed, a fixed end 201C of the suture can be held in place by the pressure between the anchor and the tissue socket (e.g. bore or socket with in the bone) in which the anchor is placed. Hence, the first or last placed anchor can include a feature or mechanism (fixed end 201C) which prevents the suture from being pulled free once it is inserted.

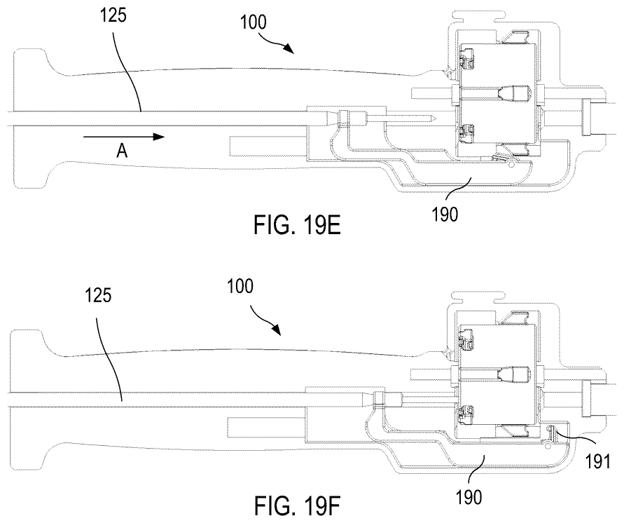

FIGS. 17A and 17B depict aspects of cartridge suture routing configurations, according to embodiments of the present invention. As shown here, a suture 200 can be routed in a cartridge 180. The suture can include a free end 205. Suture section 200S can be routed to inserted anchors and a fixed end.

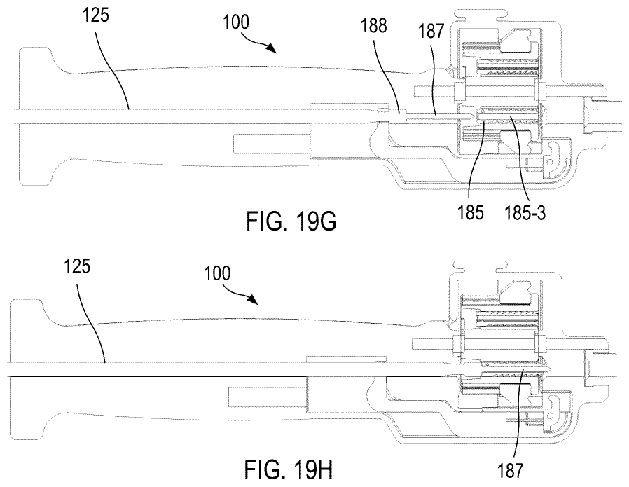

FIGS. 18A to 18D depict aspects of an anchor loading/placing procedure, according to embodiments of the present invention. As shown in FIG. 18A, a process can include setting the device 100 to an initial position, where a first anchor (not shown) is pre-loaded (e.g. within the outer sheath 140). As shown in FIGS. 18A and 18B, the knob 120 and shaft 125 can be advanced distally in the direction indicated by arrow A so as to place the first anchor 185-1 (e.g. as the first anchor 185-1 is advanced distally from the distal section of the outer sheath 140).

As shown in FIGS. 18C and 18D, the knob 120 and shaft 125 can be retracted proximally in the direction indicated by arrow B and the next anchor (e.g. intermediate anchor 185-2) can be prepared for loading. In FIG. 18D, the knob 120 and shaft 125 have been advanced distally and the second anchor 185-2 is loaded and placed. This process can be repeated until all desired anchors are placed.

FIGS. 19A to 19I depict detailed aspects of an anchor loading/placing procedure, according to embodiments of the present invention. As shown in FIG. 19A, the device 100 includes a wall 101 that prevents rotation of the cartridge 180 while the rotation stop 186 is in place.

As shown in FIG. 19B, after the anchor is placed, the shaft 125 can be drawn back proximally in the direction indicated by arrow B until the resetting arm 190 is engaged with the rotation stop 186. As the shaft 125 is drawn back proximally even further, thus drawing back proximally the resetting arm 190 even further, the resetting arm 190 can draw back proximally the rotation stop 186 with which it is engaged.

As shown in FIG. 19C, the rotation stop 186 is drawn back proximally until it reaches a gap in the wall 101 against which it is engaged. As shown in FIG. 19D, the cartridge 180 can then be rotated as indicated by arrow A (e.g. via a spring loaded mechanism), whereby the previous rotation stop (not shown) now passes through the gap in the wall and the next rotation stop 186A engages against the wall. In some embodiments, one or more springs are located on the sides of the cartridge 180 (e.g. on the proximal side and on the distal side). In some embodiments, a spring can be a torsion spring having two ends, where one end of the torsion spring is connected with the cartridge 180 and the other end of the spring is connected with the handle. In some embodiments, one or more springs can be located on a proximal side of the cartridge 180. In some embodiments, one or more springs can be located on a distal side of the cartridge 180. With reference to FIGS. 8 and 9, a cartridge 180 can include a connection mechanism 177 that is configured to couple with a torsion spring.

As shown in FIGS. 19E and 19F, the shaft 125 can be driven forward distally again. FIG. 19E shows distal movement of the shaft 125 in the direction indicated by arrow A. This can cause the resetting arm 190 to return to its original position. As shown here, the resetting arm 190 includes a hinged catch 191 that can engage the stop when moving in the proximal direction and pivot and slide past the stop when moving in the distal direction.

As shown in FIGS. 19G to 19I, as the shaft 125 is driven forward distally, the driver 188 engages with the anchor 185 and the awl 187 passes through the cannula 185-3 of the anchor 185. The shaft 125 can be driven forward distally until the anchor 185 is deployed. In some instances, an anchor connection is located on the distal end of the shaft 125, with the awl 187 extending from it. In some cases, the driver 188 can be referred to as an anchor connection.

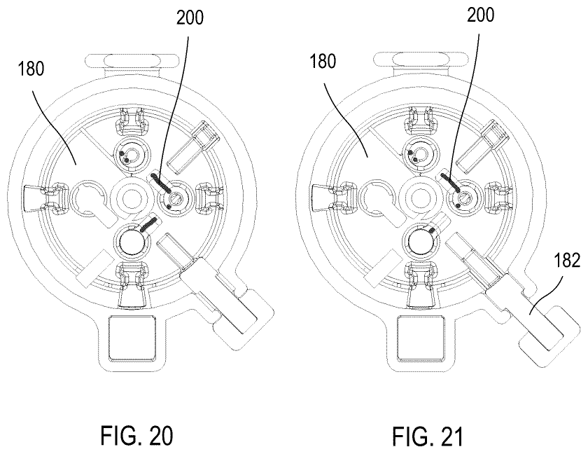

As shown in FIGS. 20 and 21, the suture routing pin 182 can be released, freeing the suture 200 from the now empty section of the cartridge 180.

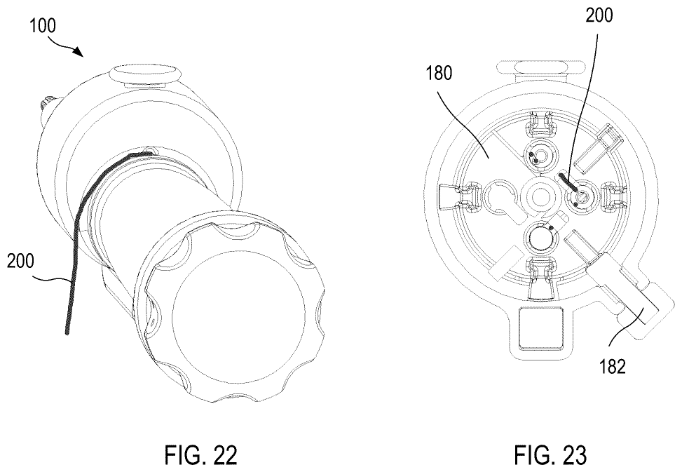

As shown in FIGS. 22 and 23, the free end of the suture 200 can be pulled, straightening the suture in the cartridge 180. The suture routing pin 182 can be returned to its earlier position, now free of the suture.

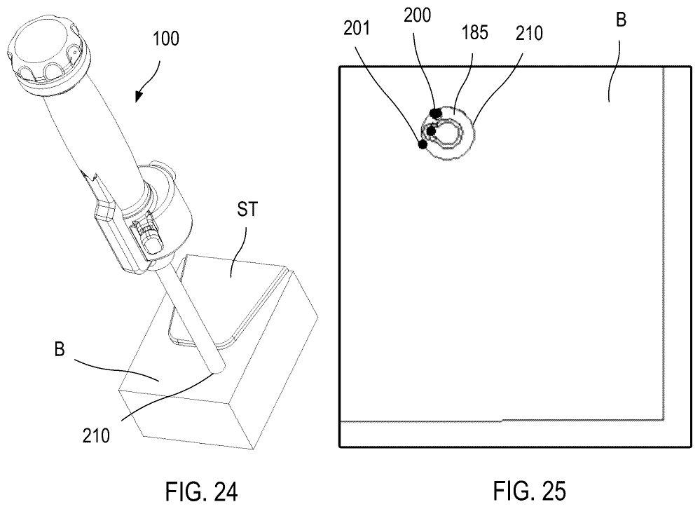

FIGS. 24 and 25 depict aspects of a surgical procedure, according to embodiments of the present invention. In some cases, the surgical procedure involves an implantation process. As shown here, a device 100 can be used to place an anchor 185 at a location within the bone B of a patient. The bone B may be near or adjacent to soft tissue ST. In some cases, the surgical procedure can involve fixing a graft or an implant to the patient in place of the soft tissue. In some cases, the surgical procedure can include fixing a graft or an implant in combination with the soft tissue. When the anchor 185 is placed, a fixed end 201 of the suture can be held in place by the pressure between the anchor 185 and the tissue socket 210 (e.g. bore or socket with in the bone) in which the anchor 185 is placed. Hence, the first placed anchor can include a feature or mechanism (fixed end 201) which prevents the suture from being pulled free once it is inserted. In some cases, anchor 185 can be referred to as the first anchor.

As illustrated in FIGS. 26 and 27, the fixation device 100 can be moved to the next position, with trailing suture 200 connected to the first anchor 185. The outer sheath 140 is placed at the desired placement location, and the second anchor 185-2 is placed. The suture 200 now extends from the first anchor 185 through the second anchor 185-2 and into the fixation device 100.

As illustrated in FIGS. 28 and 29, the fixation device 100 can be used to repeat the process until all anchors (e.g. anchors 185, 185-2, and 185-3) except for the last anchor have been placed. The device 100 can be moved to the position 218 of the final anchor, and the free end 250 of the suture 200 can be used to tension the entire length of suture (e.g. by drawing or pulling on the free end 250).

As shown in FIGS. 30 to 32, after the first three anchors have been placed (e.g. first anchor 185, first intermediate anchor 185-2, second intermediate anchor 185-3) the final anchor 185-4 can be placed while the suture is drawn taught or is otherwise in a tense or tensioned configuration. The final anchor 185-4 can operate to lock the running suture under tension once placed. For example, when the anchor 185-4 is placed, a fixed end of the suture can be held in place by the pressure between the anchor 185-4 and the tissue socket (e.g. bore or socket with in the bone) in which the anchor 185-4 is placed, similar to the suture fixed end described above in relation to FIG. 25. The fixation device 100 can be removed or withdrawn, and excess suture on the free end can be cut or removed.

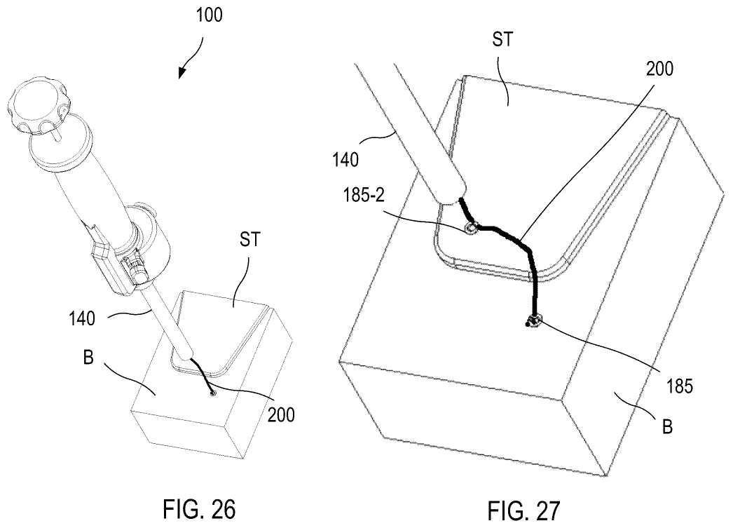

FIG. 33 depicts aspects of a lateral graft fixation device 1000, according to embodiments of the present invention. As shown here, device 1000 includes a knob 1100, a primary shaft 1200, a handle 1300, and an outer sheath 1400.

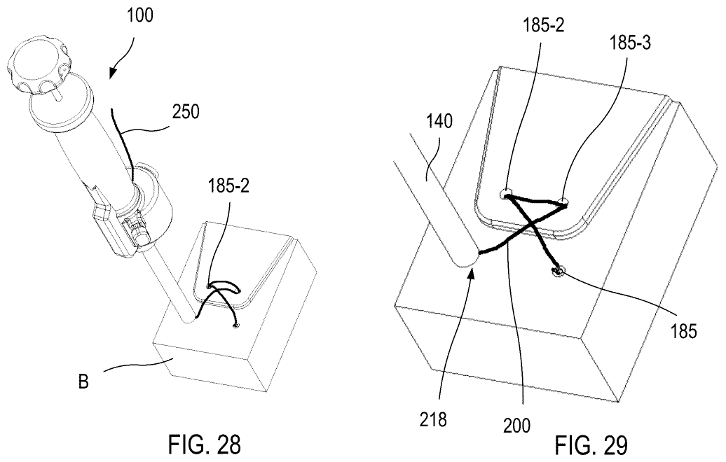

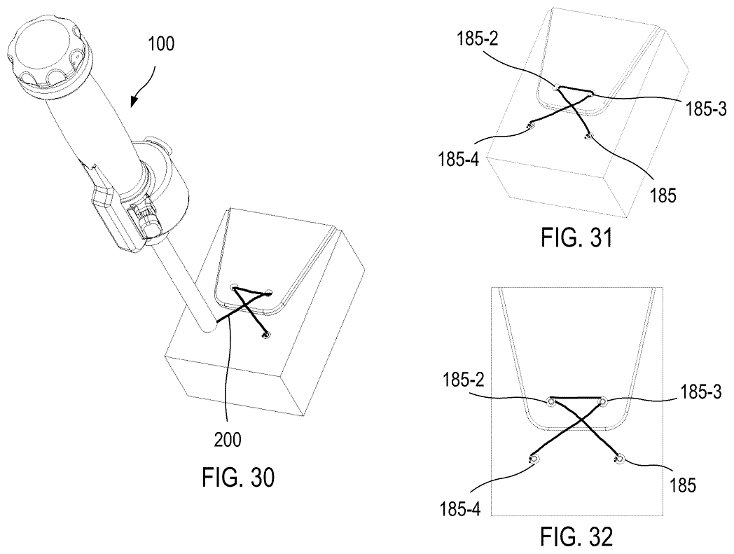

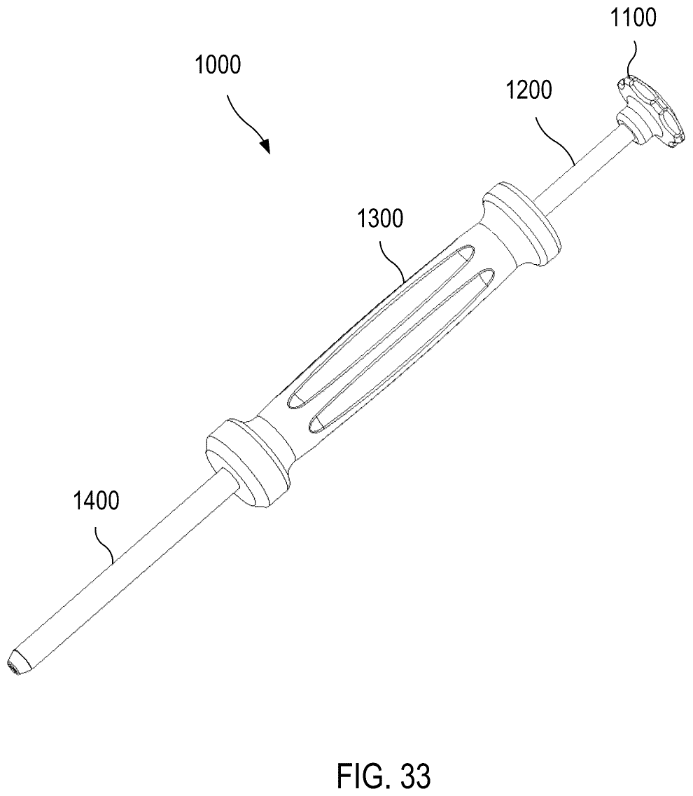

FIGS. 34 to 36 depict aspects of a lateral graft fixation device 1000, according to embodiments of the present invention. As shown here, device 1000 includes a knob 1100, a primary shaft 1200, a handle 1300, and an outer sheath 1400. The knob 1100 includes a proximal striking surface 1110. Disposed within the outer sheath 1400, the device 1000 includes an insertion shaft 1500, an awl 1600, a joining suture 1700, and an expanding anchor 1800. In operation, the primary shaft can engage with each of the internal shafts (e.g. insertion shaft 1500 and awl 1600) one at a time, or sequentially.

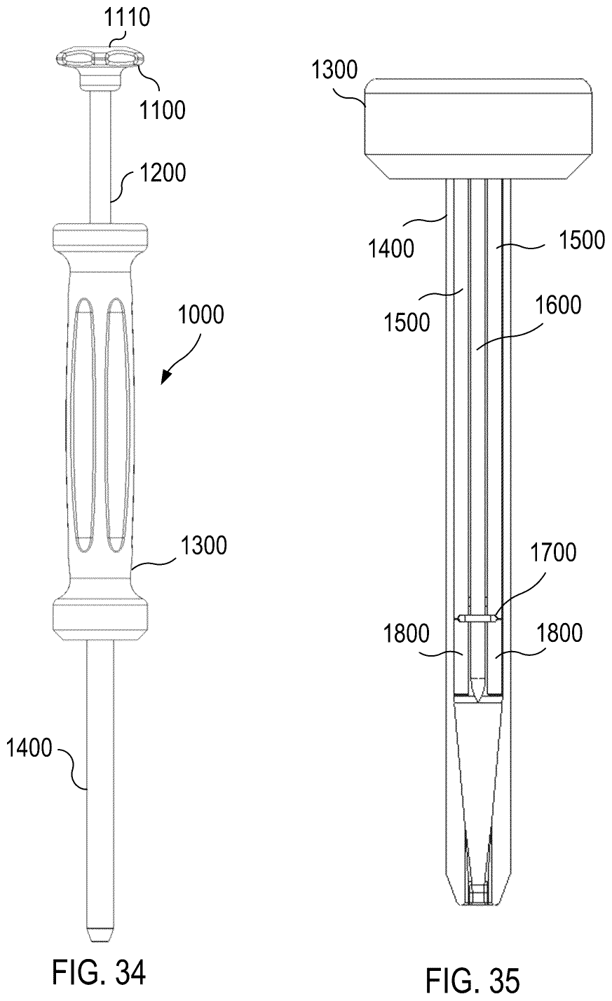

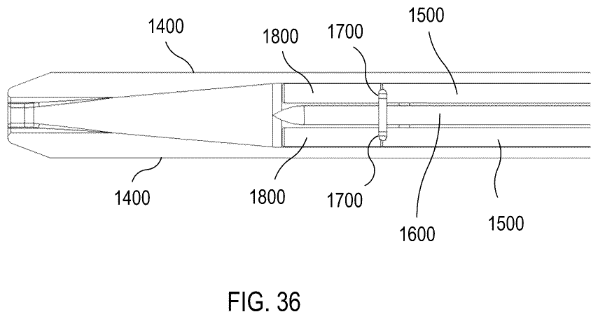

As shown in FIGS. 37 and 38, each time the primary shaft 1200 is advanced (e.g. by advancing the strike surface 1110 or otherwise moving the shaft 1200 in a distal direction relative to the handle 1300 as indicated by arrow A), one of the internal shafts is extended out of the distal section 1410 of the outer sheath 1400.

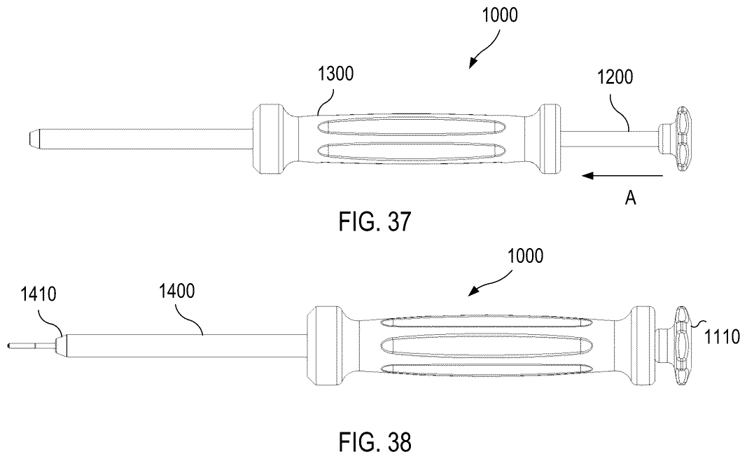

FIG. 39 depicts aspects of an implant 2000 according to embodiments of the present invention. As shown here, implant 2000 can include one or more expanded anchors 1800, a joining suture 1700, and a graft 1900. As shown here, the graft is disposed on a surface of a patient cuff C, which in turn is disposed on a surface of a patient bone B. Anchors 1800 have been inserted into bore holes 2010 and expanded therein.

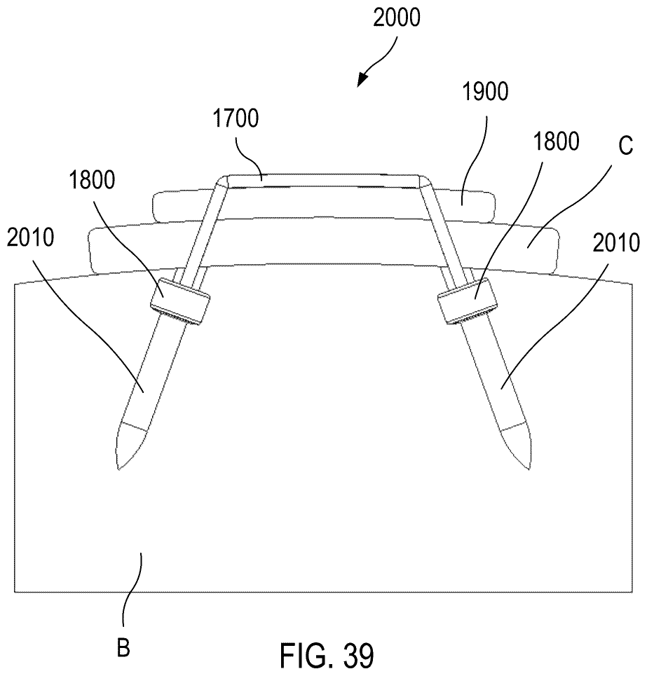

As illustrated in FIGS. 40 and 41, the outer sheath 1400 can be pressed against one side of the graft 1900 on the lateral side, and the awl 1600 can be advanced distally into the bone B by advancing the knob (not shown) distally. The awl 1600 can then be retracted proximally by pulling the knob back proximally. In this way, the device can be used to create a bore or hole 2010 in the bone B.

As illustrated in FIGS. 42 and 43, the first insertion shaft 1500 and the first expandable anchor 1800 can be inserted into the hole 2010 which was awled or bored by advancing the knob. The anchor 1800 can then be expanded, and the insertion shaft 1500 can be retracted, leaving the anchor 1800 in place in the bore 2010. In FIG. 42, the anchor 1800 is in a pre-expanded state. In FIG. 43, the anchor 1800 is in an expanded state. In some embodiments, an anchor 1800 can be expands radially when it is compressed axially. In some cases, the insertion shaft 1500 can operate to compresses the anchor 1800 axially, causing the radial expansion. For example, the insertion shaft 1500 can include a compression mechanism 1510 that can operate to compress the anchor 1800.

As illustrated in FIGS. 44 and 45, the device can be moved to the other side of the lateral side of the graft 1900, trailing the joining suture 1700, which is attached to the anchor 1800 which was just placed. The next hole or bore 2020 can be awled or created with the awl 1600 once the device is in place. As illustrated in FIGS. 46 and 47, a second anchor 1810 can be placed using a process similar to the process which was used to place the first anchor 1800.

As depicted in FIG. 48, the second anchor 1810 can be expanded, the joining suture 1700 can be tightened, and the device can be removed, leaving the implant 2000 in place.

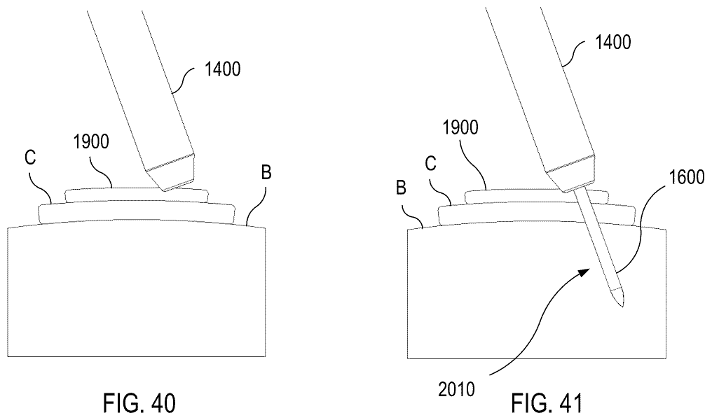

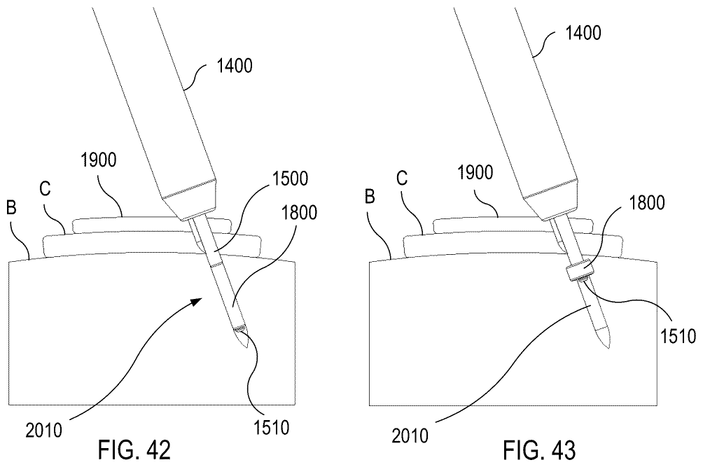

FIG. 49 depicts a distal portion of a device 1000 when in an initial position or configuration. As shown here, the device includes an outer sheath 1400. Disposed within the outer sheath 1400, the device 1000 includes a first insertion shaft 1500A and a second insertion shaft 1500B, an awl 1600, a joining suture 1700, and a first expanding anchor 1800A and a second expanding anchor 1800B. In operation, the primary shaft can engage with each of the internal shafts (e.g. insertion shafts 1500A, 1500B and awl 1600) one at a time, or sequentially. As shown in FIG. 50, the awl 1600 can be extended from the outer sheath 1400 in a distal direction as indicated by arrow A (e.g. by exiting from or extending through a distal aperture 1420 of the outer sheath 1400.

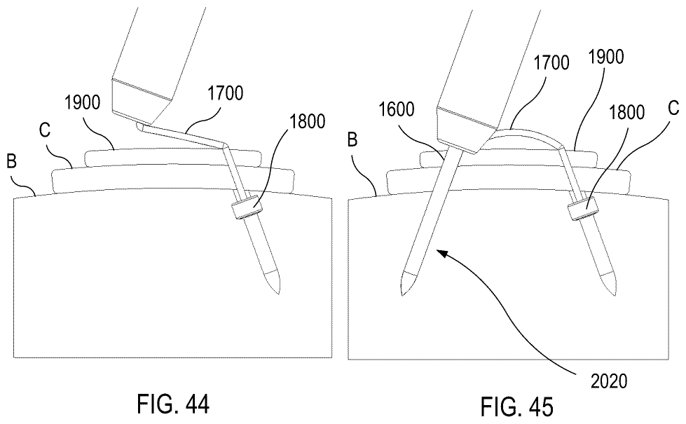

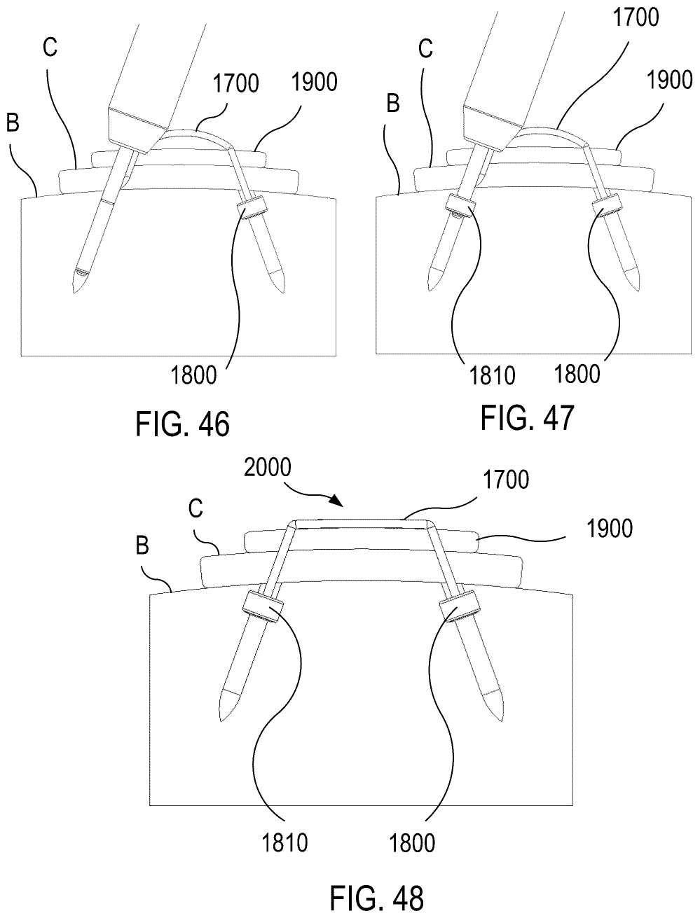

As illustrated in FIG. 51, the awl 1600 can then be retracted in a proximal direction, and a first insertion shaft 1500A can be extended in a distal direction, so as to advance a first anchor 1800A in a distal direction. As shown in FIG. 52, an expandable portion 1805A of the expandable anchor 1800A has been expanded. In some embodiments, the anchor 1800A expands radially when it is compressed axially. In some cases, the insertion shaft 1500A includes an expansion mechanism 1510A which operates to expand the anchor 1800A. For example, the expansion mechanism 1510A can be a compression mechanism that compresses the anchor axially, causing the radial expansion.

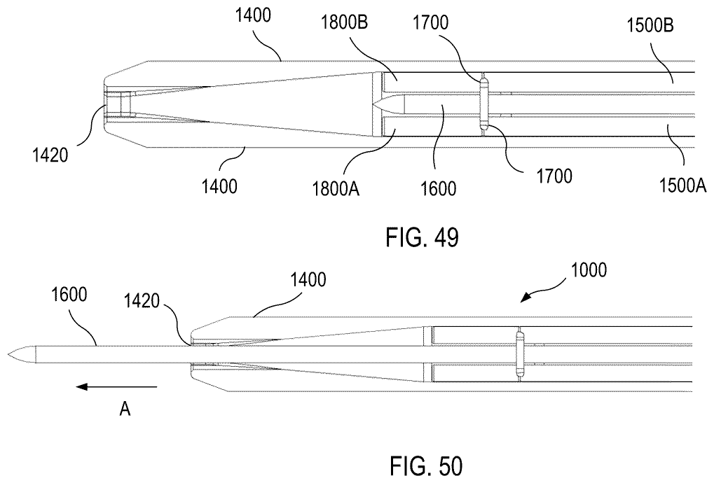

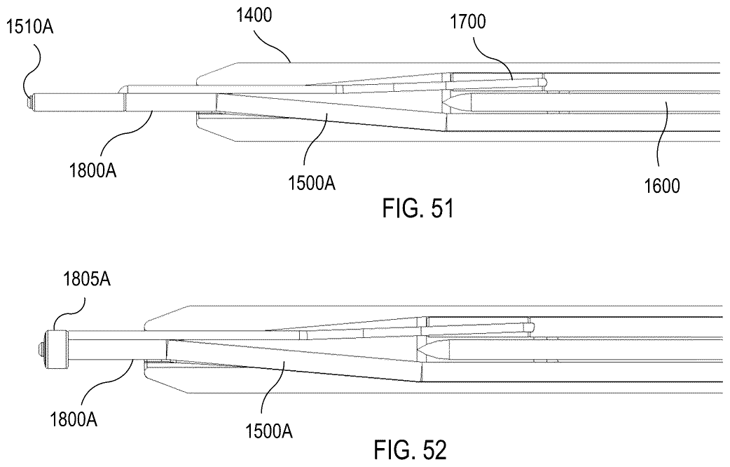

As illustrated in FIG. 53, the first insertion shaft (not shown) can be retracted in a proximal direction and the device can be repositioned, and as illustrated in FIG. 54, the awl 1600 can be extended again in the distal direction (e.g. to create another hole or bore in the patient tissue).

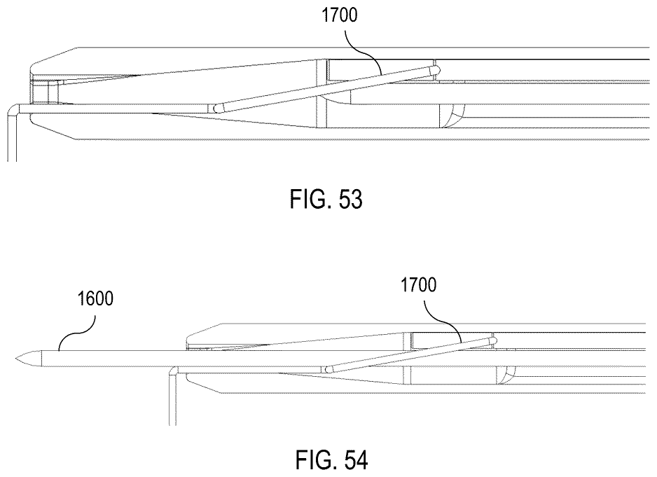

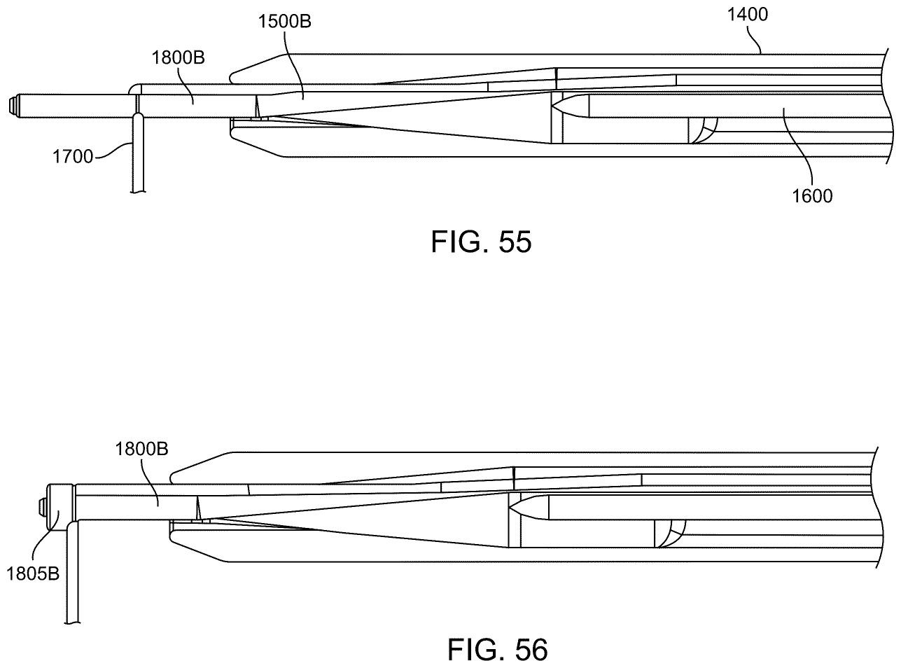

As shown in FIG. 55, the awl can be retracted in the proximal direction, and the second insertion shaft 1500B can be extended in a distal direction, so as to advance a second anchor 1800B in a distal direction. As shown in FIG. 56, the second expandable anchor 1800B can be placed, and an expandable portion 1805B of the second expandable anchor 1800B can be expanded. The device can then be removed.

Although the preceding description contains significant detail in relation to certain preferred embodiments, it should not be construed as limiting the scope of the invention but rather as providing illustrations of the preferred embodiments.

Embodiments of the present invention encompass kits having one or more components of a system as disclosed herein. In some embodiments, the kit includes one or more system components, along with instructions for using the component(s) for example according to any of the methods disclosed herein.

All features of the described systems and devices are applicable to the described methods mutatis mutandis, and vice versa.

In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for treating a bone or soft tissue of a patient, the system comprising:
a graft;
an anchor assembly that includes a first anchor, a second anchor, and a suture in operative association with the first anchor and the second anchor; and
a deployment device configured to deliver the anchor assembly to the bone or soft tissue of the patient.

2. The system according to claim 1, wherein the deployment device is configured to store the anchor assembly prior to delivering the anchor assembly to the bone or soft tissue of the patient.

3. The system according to claim 1, wherein the deployment device comprises a sheath that is configured to remain in an incision of the patient until each anchor of the anchor assembly is placed in the bone or soft tissue of the patient.

4. The system according to claim 1, wherein the anchor assembly comprises a third anchor and a fourth anchor, and wherein the suture is in operative association with the third anchor and the fourth anchor.

5. The system according to claim 1, wherein at least one anchor of the anchor assembly does not pass through the graft.

6. The system according to claim 1, wherein at least one anchor of the anchor assembly is an expandable anchor.

7. The system according to claim 1, wherein at least one anchor of the anchor assembly comprises polyether ether ketone.

8. The system according to claim 1, wherein at least one anchor of the anchor assembly is an all-suture anchor.

9. The system according to claim 1, wherein the deployment device comprises a strike surface configured for malleting.

10. The system according to claim 1, wherein the deployment device comprises a spring loaded cartridge.

11. The system according to claim 1, wherein the deployment device comprises an awl.

12. The system according to claim 1, wherein each anchor of the anchor assembly is configured to be inserted on an awl, or wherein each anchor of the anchor assembly is self-awling.

13. The system according to claim 1, wherein each anchor of the anchor assembly is configured to be stored in a revolving barrel.

14. The system according to claim 1, wherein the deployment device comprises a first insertion shaft and a second insertion shaft that is parallel with the first insertion shaft, and wherein the first anchor is configured to releasably engage with the first insertion shaft and the second anchor is configured to releasably engage with the second insertion shaft.

15. An anchor assembly for treating a bone or soft tissue of a patient, the anchor assembly comprising:
an initial anchor;
a suture affixed with the initial anchor;
one or more intermediate anchors configured to receive the suture therethrough; and
a final anchor configured to lock the suture under tension once placed.

16. The anchor assembly according to claim 15, wherein the initial anchor comprises a suture path, one or more outer fixation mechanisms, a cannula, and a driver connection mechanism.

17. The anchor assembly according to claim 15, wherein at least one of the intermediate anchors comprises a suture path, one or more outer fixation mechanisms, a cannula, and a driver connection mechanism.

18. The anchor assembly according to claim 15, wherein the final anchor comprises a suture path, one or more outer fixation mechanisms, a cannula, and a driver 2 connection mechanism.

19. The anchor assembly according to claim 15, wherein at least one of the anchors is an expandable anchor.

20. The anchor assembly according to claim 15, wherein the initial anchor and the final anchor are expandable anchors.
